(19) United States
(12) Patent Application Publication
(10) Pub. No.: US 2023/0104874 A1
(43) Pub. Date: Apr. 6, 2023

(54) HERBAL PREPARATION FOR PREVENTION AND TREATMENT OF HELICOBACTER PYLORI INFECTION

(71) Applicant: HerbElixa DOO BEOGRAD- NOVI BEOGRAD, Belgrade (RS)

(72) Inventors: Natasa Simin, Novi Sad (RS); Marija Lesjak, Sremska Kamenica (RS); Ivana Beara, Novi Sad (RS)

(21) Appl. No.: 17/904,485

(22) PCT Filed: Oct. 16, 2020
(86) PCT No.: PCT/RS2020/000016

§ 371 Date: Aug. 18, 2022

(51) Int. Cl. A61K 36/53; A61K 31/05; A61K 31/015; A61K 31/01; A61K 31/045; A61K 31/352; A61K 47/44; A61K 47/02; A61P 31/04; A61K 9/48; A61K 31/7048; A61K 31/43

(52) U.S. Cl. CPC A61K 36/53; A61K 31/05; A61K 31/015; A61K 31/01; A61K 31/045; A61K 31/352; A61K 47/44; A61K 47/02; A61P 31/04; A61K 9/4825; A61K 31/7048; A61K 31/43; A61K 2236/331

(57) ABSTRACT

The invention relates to the use of herbal preparation, characterized by presence of 4 dominant compounds (carvacrol from about 35% to about 50%, y-terpinene from about 10% to about 30%, thymol from about 10% to about 25%, p-cymene from about 8% to about 20%, as expressed in % of total peak area in GC/MS chromatogram), obtained by mixing at least two essential oils from species selected from the group of genera Satureja L., Origanum L. and Thymus L., to prepare food supplement or medicament for treatment and prevention of gastric Helicobacter pylori infection in humans The mixture may be placed on liquid or solid carrier and capsulated in non gastro-resistant capsules. The invention also relates to dosage regiment of herbal preparation for treatment and prevention of patients with Helicobacter pylori infection which leads to successful eradication of bacteria without any side-effects.

### TECHNICAL FILED

The present invention relates to a therapeutic application of a herbal preparation with particular chemical composition, obtained by mixing two or more essential oils from genera Satureja L., Origanum L. and Thymus L., to prepare a food supplement or medicament successful in eradication of pathogenic bacteria. More particularly, the invention relates to the use of this herbal preparation to prepare a food supplement or medicament successful in eradication of Helicobacter pylori (H. pylori) in humans.

### BACKGROUND OF THE INVENTION

H. pylori inhabits human stomach where it can induce many severe gastrointestinal disorders, such as gastritis, peptic and duodenal ulcers and gastric carcinoma. World Health Organization (WHO) indicates that approximately 50% of the world's population is infected with H. pylori, with different prevalence in developed (25 to 50%) and developing countries (70 to 90%). Although most of the patients never experience symptoms of disease, significantly high percentage of the infected subjects will develop peptic ulcers (approximately 10 to 20%), one-quarter of these patients (approximately 4.25%) will have serious ulcer complications, while 1 to 2% will progress to gastric cancer. Gastric cancer is the second most common cause of cancer-related deaths in the world (Kusters et al. (2006) Pathogenesis of H. pylori infection. Clin Microbiol Rev 19: 449-490). Correspondingly, H. pylori was declared as a carcinogen for humans by the WHO in 1994. Even more, due to H. pylori infection, approximately 500 million people worldwide will sooner or later suffer from severe gastric pathology i.e. peptic ulceration and approximately 30 million might progress to gastric cancer.

There is a great need to achieve eradication of H. pylori from the gastric mucosa. To this end, numerous antibiotic therapies have been used: amoxicillin, tetracycline, metronidazole, furazolidone, levofloxacin and clarithromycin (Wannmacher (2011) Review of the evidence for H. pylori treatment regimens, 18^{th} Expert Committee on the Selection and Use of Essential Medicines). Currently, therapy for H. pylori infection consists of high dosages of three to four different antibiotics, in addition to adjunctive agents. However, this therapy exhibits highly limited efficacy mainly due to extremely frequent occurrence of antibiotic-resistant strains of H. pylori. As an example: triple therapy using clarithromycin and amoxicillin/metronidazole with proton pump inhibitor (PPI) has shown success rates of 84% and 76%, while quadruple therapy consisting of a PPI, bismuth, metronidazole and tetracycline showed success rate of 87%. As even 20% of patients with H. pylori infection is not cured after completing the first course of antibiotic treatment, additional treatment cycle is required. Thus, the WHO included H. pylori in “Global priority list of antibiotic-resistant bacteria to guide research, discovery and development of new antibiotics”, which was developed by a group of 70 world-leading experts in 2017. Moreover, all mentioned treatments are often associated with severe gastrointestinal side effects which patients find hard to bear such as diarrhea, nausea, vomiting, bloating and abdominal pain.

Furthermore, several alternative therapies for eradication of H. pylori infection in humans based on microorganisms, peptides, polysaccharides and intragastric violet light irradiation have been suggested, but have not been effective in eradicating the bacteria (Ayala et al. (2014) Exploring alternative treatment for Helicobacter pylori infection, Word J Gastroenterol 20(6): 1450-1469). Additionally, recent study on vaccines confirmed that promising vaccine candidates have not been found yet (Sutton & Boag (2019) Status of vaccine research and development for Helicobacter pylori, Vaccine 37: 7295-7299).

Probiotics and some plant based dietary supplements are used to prevent or reduce some of the antibiotic therapy side-effects. However, there are no commercially available dietary supplements or other herbal products that are tested in humans to have anti-Helicobacter activity, improve the therapeutic response to antibiotics or prevent reoccurrence of H. pylori infection in humans.

Essential oils obtained from aromatic plants are well-known antimicrobial agents against many bacterial strains. Essential oils are complex mixtures of volatile organic compounds (predominantly terpenes and phenyl propanes) obtained by hydrodistillation or steam distillation from aromatic plants. Essential oil from one aromatic plant can contain several hundred different components. The chemical composition of essential oil of particular plant species is highly variable, both in qualitative and in quantitative terms. Various factors are responsible for this variability, such as: intrinsic factors related to the plant, interaction of the plant with the environment (soil type and climate, etc.), the maturity of the plant, plant organ chosen for essential oil isolation, harvest time during the day and extrinsic factors related to the extraction method. Antimicrobial potential of essential oil is highly dependent on its chemical composition. Correspondingly, the strength of antimicrobial activity of essential oil from particular plant cannot be claimed without determining its chemical profile. Therefore, when potential application of particular essential oils is investigated, chemical composition of each sample of essential oil should be defined.

Some essential oils express antimicrobial activity in in vitro bioassays even against multi-resistant H. pylori strains (Martin & Ernst (2003) Herbal medicines for treatment of bacterial infections: a review of controlled clinical trials. J Antimicrob Chemother 51: 241-246; Ohno et al. (2003) Antimicrobial activity of essential oils against Helicobacter pylori. Helicobacter 8: 207-215; Takeuchi et al. (2014) Natural products and food components with anti-Helicobacter pylori activities, World J Gastroenterol 20: 8971-8978).

There are numerous in vitro evidences on anti-H. pylori activity of essential oils obtained from species from genera Satureja L., Origanum L. and Thymus L.

For example, essential oils of Satureja bachtiarica, Satureja montana and Satureja hortensis exhibit high anti-H. pylori activity in vitro (Bergonzelli et al. (2003) Essential oils as components of a diet-based approach to management of Helicobacter infection. Antimicrob Agents Chemother 47: 3240-324; Falsafi et al. (2015) Chemical composition and anti-Helicobacter pylori effect of Satureja bachtiarica Bunge essential oil. Phytomed 22: 173-177; Lesjak et al. (2016): Binary and tertiary mixtures of Satureja hortensis and Origanum vulgare essential oils as potent antimicrobial agents against Helicobacter pylori. Phytother Res 30: 476-484).

Essential oil obtained from Origanum vulgare has shown anti-H. pylori activity in in vitro bioassays (Bergonzelli et al. (2003) Essential oils as components of a diet-based approach to management of Helicobacter infection. Antimicrob Agents Chemother 47: 3240-324). It is also proven that carvacrol, a major component of oregano essential oil, shows bactericidal activity against H. pylori strains in vitro (Marinelli et al. (2018) Carvacrol and its derivatives as antibacterial agents. Phytochem Rev 17: 903-921; Ruiz-Rico et al. (2020) In vitro antimicrobial activity of immobilised essential oil components against Helicobacter pylori. World J Microbiol Biotechnol 36: 3-9).

It has been shown that thyme (Thymus vulgaris) essential oil has antibacterial effect against H. pylori (Esmaeili et al. (2012): Anti-Helicobacter pylori activities of soya powder and essential oils of Thymus vulgaris and Eucalyptus globulus. Open Microbiol J 6: 65-69). Also, some other Thymus species express anti-H. pylori activity (Dandlen et al. (2011): Antimicrobial activity, cytotoxicity and intracellular growth inhibition of Portuguese Thymus essential oils. Rev. Bras. Farmacogn 21: 1012-1024). Additionally, a major component of thyme essential oil—thymol—showed high bactericidal activity against H. pylori strains in vitro (Korona-Glowniak et al. (2020) The in vitro activity of essential oils against Helicobacter pylori growth and urease activity. Molecules 25: 1-15).

Furthermore, it has been shown that cyclic hydrocarbons p-cymene, which is present in high amounts in thyme essential oil, and γ-terpinene, abundantly present in Satureja hortensis essential oil, have no antibacterial activity against Gram negative bacteria (Korona-Glowniak et al. (2020) The in vitro activity of essential oils against Helicobacter pylori growth and urease activity. Molecules 25: 1-15). However, it has been reported that some compounds with no antibacterial activity in the presence of other antimicrobial agents show a synergistic effect. For example, the presence of p-cymene in essential oil along with carvacrol may enhance the antimicrobial activity of the oil (Ultee et al. (2002) The phenolic hydroxyl group of carvacrol is essential for action against the food-borne pathogen Bacillus cereus. Appl Environ Microbiol 68: 1561-1568).

Moreover, mixtures of essential oils, particularly binary and tertiary mixtures of Satureja hortensis, Origanum vulgare subsp. vulgare and Origanum vulgare subsp. hirtum essential oils were more effective with respect to activity of each single oil from the mixture (Lesjak et al. (2016): Binary and tertiary mixtures of Satureja hortensis and Origanum vulgare essential oils as potent antimicrobial agents against Helicobacter pylori. Phytother Res 30: 76-484).

Concerning in vivo evidences on experimental animals, it was proven that the mixture of Satureja hortensis and Origanum vulgare subsp. hirtum essential oils successfully eradicated H. pylori in 70% of the mice without use of antibiotics, while did not express toxicity or altered cytokine and chemokine balance (Harmati et al. (2017): Binary mixture of Satureja hortensis and Origanum vulgare subsp. hirtum essential oils: in vivo therapeutic efficiency against Helicobacter pylori infection. Helicobacter e12350).

However, there are no studies present in the prior art conducted on humans, with the aim to assess the therapeutic efficacy of essential oils obtained from Satureja sp., Origanum sp. and Thymus sp., their mixtures, or pure compounds isolated from these oils, in eradication of H. pylori.

The above brief review clearly illustrates a great need for finding new therapies for eradication of H. pylori infection in humans and the various approaches suggested to combat this bacterium.

### TECHNICAL PROBLEM

The object of the present invention is to provide a herbal preparation of defined chemical profile comprising mixture of essential oils which is highly effective in eradication of H. pylori infection in humans.

The second object of the present invention is to provide a method of preparing the described herbal preparation.

The third object of the present invention is to establish dosage regiment for treatment with the herbal preparation of patients with confirmed H. pylori infection which leads to successful eradication of H. pylori from gastric mucosa.

The fourth object of the present invention is to establish a treatment with the herbal preparation for eradication of H. pylori from gastric mucosa without utilizing any of the known drugs or antibiotics.

The fifth object of the present invention is to establish a treatment with the herbal preparation for eradication of H. pylori from gastric mucosa, which does not cause any unwanted side effects, such as diarrhea, nausea, vomiting, bloating and abdominal pain, which are very common for standard antibiotic therapy.

### DISCLOSURE OF INVENTION

The invention is application of herbal preparation—a particular mixture of essential oils—as a food supplement or medicament for prevention and treatment of Helicobacter pylori infection in humans. The mixture is characterized by the presence of 4 dominant compounds in the following quantities: carvacrol from about 35% to about 50%, γ-terpinene from about 10% to about 30%, thymol from about 10% to about 25%, p-cymene from about 8% to about 20%, as expressed in % of total peak area in GC/MS chromatogram. The mixture can be obtained by mixing two or more essential oils from species from genera Satureja L., Origanum L. and Thymus L. The mixture may be placed on liquid or solid carrier and capsulated in non gastro-resistant capsules. The preparation, as disclosed in the present application, is suitable for use as a food supplement or as a herbal medicine for treatment of H. pylori infection. The dosage regiment for successful eradication H. pylori from gastric mucosa in humans is also disclosed in the present application.

### Method for Obtaining the Preparation (Mixture of Essential Oils)

The herbal preparation of the present invention must have particular chemical profile to achieve high anti-H. pylori activity. It is characterized by presence of four dominant compounds: carvacrol from about 35% to about 50%, γ-terpinene from about 10% to about 30%, thymol from about 10% to about 25%, p-cymene from about 8% to about 20%. The quantities are expressed in % of total area of all peaks in chromatogram obtained by gas chromatography with mass spectrometry detection (GC-MS). Additionally, the preparation can contain minor compounds, such as α-terpinene, trans-β-caryophyllene, α-pinene, β-pinene, β-myrcene, linalool, 1,8-cineole, limonene, each present from 0.1 to 3% (expressed in % of total area of all peaks in GC-MS chromatogram). Any other component of preparation can be present up to 0.4% each. Preferably, the composition of the preparation (expressed in % of total area of all peaks in chromatogram) should be: carvacrol from about 38% to about 48%, γ-terpinene from about 18% to about 28%, thymol from about 10% to about 12%, p-cymene from about 9% to about 14% and α-terpinene, trans-β-caryophyllene, α-pinene, β-pinene, β-myrcene, linalool, 1,8-cineole, limonene each present from 0.4 to 2.9%. The highest effect against H. pylori was obtained for the preparation with the following exact composition (expressed in % of total area of all peaks in chromatogram): carvacrol 47.5%, γ-terpinene 18.5%, thymol 11.9%, p-cymene 13.6%, α-terpinene 1.55%, trans-β-caryophyllene 1.49%, linalool 1.24%, α-pinene 0.99%, β-myrcene 0.97%, 1,8-cineole 0.55%, limonene 0.46% and β-pinene 0.40%.

The preparation, according to the present invention can be obtained by mixing two or more essential oils, preferably obtained by hydrodistillation, from the following plants: Satureja sp., Origanum sp. and Thymus sp. These plants are commonly used as spices or teas. More importantly, these essential oils are generally recognized as safe (GRAS) substances by the United States Food and Drug Administration (FDA) and have been accepted by the European Commission for their intended use as flavorings in food products.

Preferably, the herbal preparation of the present invention can be obtained by mixing essential oils of Satureja hortensis L., Origanum vulgare subsp. hirtum L. and Thymus vulgaris L. in the following ratio: 2:1:1, respectively. Since the chemical profile of essential oils is highly variable, mostly due to various ecological factors, methods of aromatic plants cultivation and methods of isolation, the preferable chemical composition of individual essential oils used to obtain the herbal preparation of the present invention is given in the table below:

TABLE
Chemical composition (% of total peak area) of
individual essential oils used for the preparation of
mixture, determined by GC-MS

| | | % of total peak area | | |
|---|---|---|---|---|
| | | Satureja | Origanum | Thymus |
| | | hortensis L. | vulgare L. | vulgaris L. |
| AI | Compound | essential oil | essential oil | essential oil |
| 925 | α-Thujene | 0.38 | 0.21 | 0.93 |
| 932 | α-Pinene | 1.22 | 0.20 | 1.76 |
| 947 | Camphene | 0.46 | 0.18 | 0.63 |
| 976 | β-Pinene | — | 0.43 | 1.55 |
| 990 | β-Myrcene | 1.32 | 0.20 | 1.40 |
| 1005 | α-Phellandrene | 0.09 | 0.03 | 0.12 |
| 1016 | α-Terpinene | 2.02 | 0.61 | 1.55 |
| 1023 | p-Cymene | 13.54 | 4.64 | 21.61 |
| 1028 | Limonene | — | 0.64 | — |
| 1030 | 1,8-Cineole | — | 1.00 | 0.79 |
| 1057 | γ-Terpinene | 30.01 | 3.88 | 7.95 |
| 1088 | α-Terpinolene | 0.30 | — | — |
| 1100 | Linalool | 0.14 | 2.05 | 3.24 |
| 1141 | Camphor | — | 0.81 | — |
| 1164 | Borneol | — | 0.74 | 1.04 |
| 1174 | Terpinen-4-ol | — | 0.53 | — |
| 1186 | α-Terpineol | | 0.60 | — |
| 1292 | Thymol | 0.29 | 1.73 | 48.26 |
| 1303 | Carvacrol | 48.97 | 77.38 | 7.92 |
| 1418 | trans-β- | 1.26 | 3.59 | 1.02 |
| | Caryophyllene | | | |
| 1452 | α-Humulene | — | 0.54 | — |

AI-arithmetic retention index;
^{a}Compounds with abundance >1% are written in bold

The essential oils disclosed in the present invention are obtained from plant species Satureja hortensis L., Origanum vulgare subsp. hirtum L. and Thymus vulgaris L. The plant material was collected in flowering stage and after air drying on 25-30° C., it was grounded in blender. Essential oils were isolated by hydrodistillation according to procedure of Ph. EUR. IV (European Pharmacopeia, 2002) in Clavenger apparatus. Briefly, 100 g of plant material was tapped with water and distillation lasted for 3 h. After cooling, pure essential oil was taken from Clavenger apparatus and dried with anhydrous sodium sulfate. After removal of the sulfate by filtration through Buchner funnel, essential oil was collected and stored at −20° C. until used. The obtained essential oils of Satureja hortensis L., Origanum vulgare subsp. hirtum L. and Thymus vulgaris L. were mixed in the following ratio: 2:1:1, respectively, to obtain the herbal preparation of the present invention which was further used for treatment of H. pylori-infected human volunteers.

### Potential Application of the Present Invention

The herbal preparation according to the present invention may be used for treatment or prevention of H. pylori infection as a food supplement or herbal medicine. If it is used as a medicine, it could be applied alone or in combination with standard antibiotic therapy. The preparation (the particular mixture of essential oils) can be placed on liquid or solid carrier and capsulated in gelatin capsules. Each capsule must contain at least 50 mg of the preparation and preferably 65 mg of the preparation.

The efficacy of the herbal preparation of the present invention against H. pylori is proven by both in vitro and in vivo studies.

The herbal preparation of the present invention was tested against 20 H. pylori clinical strains, categorized into 4 different populations comprising HpEurope (European), HpEAsia (East Asian), HpAsia2 (Indian) and HpAfrica1 (African). Most of the strains were multiple antibiotics resistant. The tests were performed using agar dilution method. The minimal inhibitory activity (MIC) was determined as the lowest concentration of the composition required to completely inhibit H. pylori growth. Minimal bactericidal concentration (MBC) was determined by a viable colony count, and the bactericidal effectiveness of the product is expressed as a percentage reduction of bacterial colony count compared to the untreated control. The results confirmed that preparation is active at the same activity level against different antibiotics resistant H. pylori strains, meaning that the preparation of the present invention is not selective against particular H. pylori type and that the preparation has no specific cellular targets. Namely, the essential oils are complex mixtures of high number of lipophilic compounds which penetrate through the cell wall, disrupt the bacterial membrane and interfere with integral membrane proteins. This complexity of mixture completely disables the microorganism to develop resistance. This implies that preparation is suitable for eradication of all types of H. pylori, meaning that it has potential for application for treatment of patients from all over the world, no matter which strain of H. pylori (antibiotic resistant or antibiotic non-resistant) caused the infection.

Within the present patent application the results of treatment of 28 human volunteers (divided in three groups) with the herbal preparation of the present invention are disclosed.

The first group included 10 human volunteers with confirmed acute H. pylori infection with mild symptoms of infection (bloating, stomach ache, higher level of gastric acid, coughing, loss of appetite). The volunteers were taking gelatin capsules containing 65 mg of the preparation dissolved in 435 mg of sunflower oil (5 volunteers) or put on solid carrier (5 volunteers). Each volunteer was taking 2 capsules a day (65 mg of the preparation per capsule), 1 in the morning and 1 in the evening on empty stomach 30 minutes before meal, for 30 consecutive days, i.e. 130 mg of the herbal preparation daily, 65 mg twice a day for 30 consecutive days.

During these 30 days they were not receiving any other therapy for H. pylori infection, and had normal dietary regime, only without alcohol. Four and eight weeks after the end of the treatment, volunteers were subjected to H. pylori stool antigen testing. The eradication of the infection was achieved in 50% of cases. That is why we concluded that the dosage should be higher, at least at the beginning of the treatment, and longer.

The second group included 15 human volunteers with confirmed acute H. pylori infection with mild symptoms of infection (bloating, stomach ache, higher level of gastric acid, coughing, loss of appetite). In comparison with dose regimen applied in the first group dose regimen applied in second group was higher at the beginning of the treatment and longer. The volunteers were taking gelatin capsules containing 65 mg of the preparation dissolved in 435 mg of sunflower oil (7 volunteers) or put on solid carrier (8 volunteers). Each volunteer was taking 4 capsules a day, 2 in the morning and 2 in the evening on empty stomach 30 minutes before meal, for 15 consecutive days, followed by 2 capsules per day, one in the morning and one in the evening on empty stomach 30 minutes before meal, for another 30 days, i.e. 260 mg of the herbal preparation daily, 130 mg twice a day for 15 consecutive days, followed by 130 mg of the herbal preparation daily, 65 mg twice a day for another 30 days.

During these 45 days they were not receiving any other therapy for H. pylori infection, and had normal dietary regime, only without alcohol. Four and eight weeks after the end of the treatment, volunteers were subjected to H. pylori stool antigen testing. In 14 of 15 volunteers complete eradication of H. pylori infection was confirmed (the eradication of the infection was achieved in 93% of cases). Ten volunteers reported complete loss of the symptoms of disease (stomach pain, increased level of gastric acid, bloating) after 10 days from the beginning of the treatment, while other four lost the symptoms until the end of the therapy. This implies that the herbal preparation according to the present invention, beside antibacterial effect against H. pylori, expresses anti-inflammatory effect. During this treatment, none of the volunteer reported any gastrointestinal side effects, such as diarrhea, nausea, vomiting or abdominal pain, which are very common during antibiotic therapy. These results strongly confirm that the preparation of the present invention is powerful in eradication of H. pylori infection from gastric mucosa, preferably without use of antibiotics, and does not cause any unwanted side effects.

In third group 3 other volunteers with chronic infection (symptoms present more than one year) were taking standard therapy (PPI two times a day+clarithromycin (2×500 mg)+amoxicillin (2×1000 mg)) for two weeks, together with the gelatin capsules containing 65 mg of the preparation of the present invention dissolved in 435 mg of sunflower oil. Four and eight weeks after the end of the treatment volunteers were subjected to H. pylori stool antigen testing which confirmed the absence of infection. This confirms that the preparation of the present invention does not lower the effect of the standard therapy, but rather facilitate it.

Furthermore, unlikely all currently known and used therapeutic agents in treatment of infection caused by H. pylori, the preparation disclosed herein is completely natural composition and does not contain any synthetic compounds. This makes this composition preferable for people allergic or hypersensitive to antibiotics.

Also, this preparation may be used for prevention of H. pylori infection among those who are prone to gastritis and H. pylori infection. For prevention purposes, preparation could be taken once or twice during year for 60 consecutive days, one capsule a day on empty stomach 30 minutes before meal, i.e. 65 mg of the herbal preparation daily for 60 consecutive days. Preferably, this treatment should be at the end of summer or end of winter period.

The preparation of the present invention is particularly suitable for patients with proven H pylori infection, particularly for patients who prefer herbal therapy instead of using antibiotics and even more preferably for patients that were previously treated with antibiotics but not cured after completing antibiotic treatment.

1.-14. (canceled)

15. An herbal preparation comprising at least one essential oil from plant species of each genera Satureja L., Origanum L., and Thymus L., for use in treatment of gastric Helicobacter pylori infection in humans.

16. The herbal preparation according to claim 15, wherein the herbal preparation comprises:
carvacrol from about 35% to about 50%,
γ-terpinene from about 10% to about 30%,
thymol from about 10% to about 25%,
p-cymene from about 8% to about 20%,
as expressed in % of total peak area in GC/MS chromatogram.

17. The herbal preparation according to claim 16, further comprising:
a-terpinene, trans-β-caryophyllene, a-pinene, β-pinene, β-myrcene, linalool, 1,8-cineole and limonene, each present in the herbal preparation from 0.4% to 2.9%,
as expressed in % of total peak area in GC/MS chromatogram.

18. The herbal preparation according to claim 15, wherein the herbal preparation comprises:
carvacrol from about 38% to about 48%,
γ-terpinene from about 18% to about 28%,
thymol from about 10% to about 12%,
p-cymene from about 9% to about 14%, and
a-terpinene, trans-βcaryophyllene, a-pinene, β-pinene, β-myrcene, linalool, 1,8-cineole and limonene, each present in the herbal preparation from 0.4% to 2.9%, as expressed in % of total peak area in GC/MS chromatogram.

19. The herbal preparation according to claim 15, wherein the herbal preparation comprises:
47.5% carvacrol,
18.5% γ-terpinene,
11.9% thymol,
13.6% p-cymene,
1.55% α-terpinene,
1.49% trans-β-caryophyllene,
1.24% linalool,
0.99% α-pinene,
0.97% β-myrcene,
0.55% 1,8-cineole,
0.46% limonene, and
0.40% β-pinene, as expressed in % of total peak area in GC/MS chromatogram.

20. The herbal preparation according to claim 15, wherein the herbal preparation is formulated for administration twice daily in a 65 mg dosage form.

21. The herbal preparation according to claim 15, wherein the herbal preparation is formulated for administration twice daily in a 130 mg dosage form.

22. The herbal preparation according to claim 15, further comprising a liquid carrier or solid carrier.

23. The herbal preparation according to claim 22, wherein the liquid carrier is sunflower oil.

24. The herbal preparation according to claim 22, wherein the solid carrier comprises silica.

25. The herbal preparation according to claim 15, wherein the herbal preparation is administered orally.

26. The herbal preparation according to claim 15, wherein the herbal preparation is administered in the form of a capsule.

27. The herbal preparation according to claim 15, wherein the plant species are Satureja hortensis L., Origanum vulgare L., and Thymus vulgaris L.

28. The herbal preparation according to claim 15, wherein the herbal preparation comprises carvacrol, γ-terpinene, thymol, p-cymene, α-terpinene, trans-β-caryophyllene, linalool, α-pinene, β-myrcene, 1,8-cineole, limonene, and β-pinene.

29. A method for treating a subject suffering from gastric Helicobacter pylori infection, comprising administering a therapeutically effective amount of the herbal preparation of claim 1 to the subject.

30. The method according to claim 29, wherein the herbal preparation is administered in combination with one or more antibiotics.

31. The method according to claim 29, wherein the herbal preparation is formulated for administration twice daily in a 65 mg dosage form for 30 consecutive days.

32. The method according to claim 29, wherein the herbal preparation is formulated for administration twice daily in a 130 mg dosage form for 15 consecutive days followed by administration twice daily in a 65 mg dosage form for 30 consecutive days.

33. A method of preparing the herbal preparation according to claim 15, the method comprising the following steps:
collecting aerial parts of at least one plant species from each genera Satureja L., Origanum L. and Thymus L. in flowering stage to form collected plant material,
air drying the collected plant material at 25-30° C. to form dried plant material,
grounding of the dried plant material to form grounded material,
obtaining essential oils from the grounded material,
drying the essential oils with anhydrous sodium sulfate,
removal of sulfate from the essential oils by filtration, and
mixing the essential oils to form the herbal preparation of claim 15.

34. The method according to claim 33, the method further comprising:
mixing of the obtained essential oils of Satureja hortensis L., Origanum vulgare subsp. Hirtum L. and Thymus vulgaris L. in the following ratio 2:1:1, respectively.

35. The method according to claim 33, wherein obtaining essential oils comprises hydrodistillation of the ground material.
